(19) United States
(12) Patent Application Publication
(10) Pub. No.: US 2008/0200526 A1
(43) Pub. Date: Aug. 21, 2008

(54) Composition for the Prevention and Treatment of Allergic Inflammatory Disease

(76) Inventors: Jin Soo Lee, Gyeonggi-do (KR); Sae Kwang Ku, Gyeonggi-do (KR); Sang Ho Lee, Gyeonggi-do (KR); Jei Man Ryu, Gyeonggi-do (KR)

Correspondence Address: Casimir Jones, S.C., 440 Science Drive, Suite 203, Madison, WI, 53711, US

(21) Appl. No.: 11/631,762

(22) PCT Filed: Jul. 5, 2005
(86) PCT No.: PCT/KR05/02139

§ 371 Date: Apr. 18, 2008

(30) Foreign Application Priority Data
Jul. 5, 2004 (KR) 10-2004-0052071

(51) Int. Cl. A61K 31/426; A61P 29/00

(52) U.S. Cl. 514/365

(57) ABSTRACT

Disclosed herein is a composition for the treatment and prevention of allergic inflammatory diseases comprising N-hydroxy-4-{5-[4-(5-isopropyl-2-methyl-1,3-thiazol-4-yl)phenoxy]pentoxy}-benzamidine, 4-{5-[4-(5-isopropyl-2-methyl-1,3-thiazol-4-yl)phenoxy]pentoxy}-benzamidine or pharmaceutically acceptable salts. The composition exhibits excellent medicinal effects on allergic inflammatory diseases, with a great reduction in typical chronic inflammation symptoms, such as an increase of eosinophil level in bronchoalveolar lavage fluid, total leukocyte level and eosinophil level in blood, the hypertrophy or hyperplasia of bronchial epithelium due to an increase in the number of mucus cells, a reduction in alveolar surface area resulting from the hyptertrophy of alveolar walls, and the infiltration of inflammatory cells.

### TECHNICAL FIELD

The present invention relates, in general, to a composition for the prevention and treatment of allergic inflammatory diseases and, particularly, to a composition for preventing or treating allergic inflammatory diseases comprising N-hydroxy-4-{5-[4-(5-isopropyl-2-methyl-1,3-thiazol-4-yl)phenoxy]pentoxy}-benzamidine, 4-{5-[4-(5-isopropyl-2-methyl-1,3-thiazol-4-yl)phenoxy]pentoxy}-benzamidine or pharmaceutically acceptable salts thereof.

### BACKGROUND ART

As diverse pathologies associated with environmental pollution, stress, living environments, etc. has increased, so too has allergic inflammatory disease. Allergic inflammatory disease is attributed to an abnormality in the immune system where a nasal or bronchial mucosa or a skin is hypersensitive to external allergens. Basic causes of allergies include stress, extravasated blood, etc., however the major cause is nutrition imbalance.

Depending on the site where immune responses occur against exogenous allergens, allergic inflammatory disease is represented as various symptoms including allergic rhinitis, asthma, atopic dermatitis, etc. In addition, allergic conjunctivitis, allergic dermatitis, contact dermatitis, urticaria, etc. are within the scope of allergic inflammatory diseases. Since these symptoms, although very diverse, are common in the pathology based on the hypersensitivity to externally introduced matter, a suppressant of excessive immune responses can be prescribed for all of them.

Asthma, representative of allergies, is a chronic inflammatory disease occurring in the respiratory organ, especially, the lungs and the bronchi. When patients with asthma take drugs or excessive exercise or inhale contaminated and/or cold air, their respiratory organs, especially, upper respiratory organs increase in responsiveness. This hyper-responsiveness is associated with the airflow obstruction in the airway, that is, airway obstruction or tracheal stenosis, but is readily alleviated using a bronchodilator. Included in the consensus characteristics of asthma, hyper-responsiveness to indoor and/or outdoor allergens and airway contraction are known to be mediated by mast cells and eosinophil IgE (Beasley et al., Am. Rev. Respir. Dis., 129, 806-817, 1989).

Asthma is accompanied by the allergic hyper-responsiveness mainly in the bronchia and the lungs. Particularly, the air passage is clogged by the proliferation of mucous cells and the inflammation of epithelial connective tissues in the bronchia. Also, the lungs are known to show similar histological behaviors. The pathology of asthma, although not yet clearly revealed thus far, is reported to be featured by airway stenosis, edema, mucus secretion, inflammatory cell infiltration, etc. In the mechanism of a typical exogenous asthma, when an antigen is introduced into the airway, B cells produce antigen specific antibodies IgE and IgG in cooperation with macrophages and helper T-cells. These antigen specific antibodies bind to receptors on the surfaces of mast cells and basophils, which are then activated upon re-exposure to the same antigen so as to release various cytokines and mediators of allergy/inflammation, including histamine, prostaglandin D₂, slow reacting substances (leukotriene C₄, D₄), etc. out of the cells. Due to these cytokines and mediators, when exposed to aeroallergen, patients with asthma exhibit an early asthma response characterized by a rapid airway constriction over a period of seconds to minutes and apparent recovery within 30 to 60 min from the constriction. Then, the mediators secreted from mast cells and the cytokines secreted from macrophages, mast cells and helper T-cells proliferate and activate inflammatory cells, including eosinophils, to exhibit a late asthmatic response in which bronchoconstriction, mucus secretion and inflammatory cell infiltration begin 3 to 4 hours and peak 4 to 18 hours after exposure to aeroallergens (Robertson et al., J. Allergy Clin. Immunol., 54, 244-257, 1974).

Currently available therapeutic agents for the treatment of asthma include beta 2-adreno receptor agonists, which dilate airway smooth muscles and effectively inhibit the secretion of hyperresponsiveness mediators from mast cells, adrenal cortical hormones, which exhibit an immunosuppressive effect, and disodium cromoglycate and nedocromil sodium, both known to inhibit both the early and the late asthma response. However, beta 2-adreno receptor antagonists show the treatment effect only for a short period of time and allow the ready recurrence of the disease. Adrenal cortical hormones have fragmentary treatment effects, with the concomitance of serious side effects upon long-term dosage.

Studies on the blockage of actions of arachidonic acid metabolites (including prostaglandine), lipoxigenase and leukotrienes have recently been introduced as approaches to inflammation and hyperresponsiveness reduction. Leukotriene B4, one of the arachidonate metabolites formed in the 5-lipoxygenase pathway, is involved in the action of the tissue-degenerative enzyme and reactive chemicals secreted by tissue-infiltrative and -coagulative polymorphic nucleated leukocytes.

As mentioned hereinbefore, however, because many factors in addition to leukotriene B₄ are involved in the occurrence of asthma and thus can induce various responses in vivo, certain compounds, although these are effective in inhibiting leukotriene B₄, cannot be expected to have an effect on the treatment of asthma.

For instance, according to a clinical test for the indication of asthma, conducted by Lilly corporation, U.S.A. (Clint D. W. Brooks et al., J. Med. Chem. 1996 39 (14), 2629-2649), the leukotriene B₄ receptor antagonist LY293111 is reported to be ineffective in treating asthma (Evans D J, Thorax. December 1996;51(12):1178-84). Leukotriene B4 receptor antagonist ONO-4057 is also reported to have a medicinal effect on bronchial asthma when used in combination with the cysteinyl leukotriene receptor antagonist Zafirlukast, but to be ineffective for the treatment thereof when used alone (Sakurada T. et, al., Eur J Pharmacol. Apr. 9, 1999:370(2):153-9). Accordingly, it can not be said that leukotriene B4 receptor antagonists are effective for the treatment of allergic inflammatory diseases including asthma.

In the meanwhile, leukotriene B4 receptor antagonists are used for the treatment of various diseases. For example, Japanese Unexamined Patent Publication No. 6-502164 discloses novel monocyclic and bicyclic aryl compounds which are effective for the treatment of rheumatic arthritis, gout, psoriasis, and inflammatory bowel diseases by selectively inhibiting leukotriene B4. In Japanese Pat. Laid-Open Publication No. 4-244023, omega-6 unsaturated fatty acids, such as dihomo-γ-linolenic acid, are described to have medicinal effects on arrhythmia, acute myocardial infarction, with inhibitory activity against the production of leukotriene B4. Japanese Pat. Laid-Open Publication No. 1-190656 discloses novel leukotriene B3 dimethyl amide that is effective as an anti-inflammatory agent, an anti-rheumatic agent, and a gout medicament, with antagonistic activity against leukotriene B4.

Leading to the present invention, the intensive and thorough study on the treatment of allergic inflammatory diseases, conducted by the present inventors, resulted in the finding that N-hydroxy-4-{5-[4-(5-isopropyl-2-methyl-1,3-thiazol-4-yl)phenoxy]pentoxy}-benzamidine and 4-{5-[4-(5-isopropyl-2-methyl-1,3-thiazol-4-yl)phenoxy]pentoxy}-benzamidine, which had been developed as a medicament for the treatment of osteoporosis by the present inventors (Korean Pat. Laid-open Publication No. 10-2003-8654), can greatly reduce typical chronic inflammation symptoms, such as an increase of eosinophil level in bronchoalveolar lavage fluid, and total leukocyte level and eosinophil level in blood, the hypertrophy or hyperplasia of bronchial epithelium due to an increase of mucus producing cells, a reduction in alveolar surface area resulting from the hypertrophy of alveolar walls, and the infiltration of inflammatory cells, exhibiting excellent medicinal effects on allergic inflammatory diseases including asthma.

### DISCLOSURE OF THE INVENTION

Accordingly, the present invention has been made keeping in mind the above problems occurring in the prior art, and an object of the present invention is to provide a composition for the prophylaxis and treatment of allergic inflammatory diseases, comprising N-hydroxy-4-{5-[4-(5-isopropyl-2-methyl-1,3-thiazol-4-yl)phenoxy]pentoxy}-benzamidine, 4-{5-[4-(5-isopropyl-2-methyl-1,3-thiazol-4-yl)phenoxy]pentoxy}-benzamidine or pharmaceutically acceptable salts thereof, is provided.

Another object of the present invention is to provide a method of treating and preventing allergic inflammatory diseases using the composition.

### BRIEF DESCRIPTION OF THE DRAWINGS

FIG. 1 is an optical microphotograph showing sliced specimens of asthma-induced lung tissue, stained with Masson's trichrome.

### BEST MODE FOR CARRYING OUT THE INVENTION

The present invention pertains to a composition for the prevention and treatment of allergic inflammatory diseases, comprising a benzamidine compound represented by the following chemical formula 1 or a pharmaceutically acceptable salt thereof.

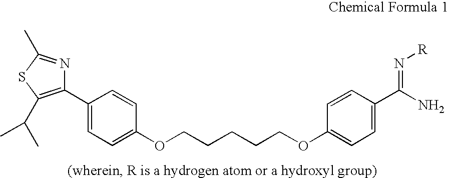

The benzamidine compound of Chemical Formula 1 may be used in the form of pharmaceutically acceptable salts known in the art. Preferable are acid addition salts prepared with pharmaceutically acceptable free acids. Free acids suitable for use in the present invention may be inorganic acids or organic acids. Examples of the inorganic acids include hydrochloric acid, bromic acid, sulfuric acid, phosphoric acid, etc, and the organic acids may be exemplified by citric acid, acetic acid, lactic acid, tartaric acid, fumaric acid, formic acid, propionic acid, oxalic acid, trifluoroacetic acid, methane sulfonic acid, benzene sulfonic acid, maleic acid, benzoic acid, gluconic acid, glycolic acid, succinic acid, 4-morpholine ethane sulfonic acid, camphorsulfonic acid, 4-nitrobenzene sulfonic acid, hydroxyl-O-sulfonic acid, 4-toluene sulfonic acid, galacturonic acid, embonic acid, glutamic acid and aspartic acid.

The benzamidine compound of Chemical Formula 1 may be prepared according to known processes (Lee, Sung-Eun, Synthesis and Biological Activity of Natural Products and Designed New Hybrid Compounds for the Treatment of LTB4 Related Disease, Busan National University, a thesis for a Ph. D degree, August 1999).

As used herein, the term “allergic inflammatory diseases” means non-specific inflammatory diseases caused by various allergens, exemplified by allergic rhinitis, asthma, allergic conjunctivitis, allergic dermatitis, atopic dermatitis, contact dermatitis, and urticaria.

In the specific embodiment of the present invention, the benzamidine compound of Chemical Formula 1 was found to have a great effect of reducing typical chronic inflammation symptoms, such as an increase of eosinophil level in bronchoalveolar lavage fluid, and total leukocyte level and eosinophil level in blood, the hypertrophy or hyperplasia of bronchial epithelium due to an increase of mucus cells, a reduction in alveolar surface area resulting from the hypertrophy of alveolar walls, and the infiltration of inflammatory cells.

The composition of the present invention may further comprise at least one effective ingredients which are equivalent or similar function to that of the benzamidine compound of Chemical Formula 1 or its pharmaceutically acceptable salt.

The composition of the present invention may further comprise one or more pharmaceutically acceptable carriers. A proper carrier may be selected from a group consisting of saline, sterilized water, Ringer's solution, buffered saline, a dextrose solution, a maltodextrin solution, glycerol, ethanol, and combinations thereof and may be, if necessary, further supplemented with other typical additives such as an antioxidant, a buffer, a static agent, etc. In combination with a diluent, a dispersant, a surfactant, a binder, and a lubricant, the composition of the present invention may also be formulated into injectable dosage forms, such as aqueous solutions, suspensions, emulsions, etc., pills, capsules, granules, and tablets. Moreover, depending on kinds of ingredients or diseases, the formulation may be conducted using methods known in the art or disclosed in Remington's Pharmaceutical Science ((the latest version), Mack Publishing Company, Easton Pa.).

According to purposes, the composition of the present invention may be administered orally or parenterally (e.g., intravenously, subcutaneously, intraabdominally, or topically). The dosage amount of the composition of the present invention varies depending on body weight, age, gender, health state, diet, administration time period, administration route, excretion rate, disease severity, etc. When account is taken of all these factors, the benzamidine compound of Chemical Formula 1 is administered once or many times at a dose of approximately 10 to 1,000 mg/kg a day and preferably at a dose of approximately 50 to 500 mg/kg a day.

For the prevention and treatment of allergic inflammatory diseases, the composition of the present invention can be used alone or in combination with surgery, hormone therapy, chemical therapy, and/or a biological response controller.

A better understanding of the present invention may be obtained through the following examples which are set forth to illustrate, but are not to be construed as the limit of the present invention.

### EXAMPLE

### Therapeutic Effect in Mouse Model of Asthma Induced with Ovalbumin

The benzamidine compound of Chemical Formula 1 was assayed for therapeutic effect on allergic inflammation in mouse models of ovalbumin-induced asthma. Starting at the sensitization with ovalbumin, the administration of the benzamidine compound was for 18 consecutive days. The experimental animals were re-exposed to ovalbumin 15 days after the sensitization and then sacrificed 3 days after the re-exposure. Changes in lung weight, cellular components of peripheral blood and bronchoalveolar lavage fluid, and lung histopathology were observed.

### 1. Experimental Animals and Breeding Management

A total of 20 female C57BL/6 mice (6-week-old, BioGenomics, Korea) was adapted to a laboratory environment for 6 days before being used in earnest experiments. While being housed at a density of five in a plastic cage, the experimental animals were bred in a breeding room with controlled temperature (20 to 25° C.) and humidity (30 to 35%). Under light-dark cycles of 12 hours, mice were allowed to have free access to feedstuff and tap water. While asthma was induced in 15 mice by ovalbumin, 5 mice were used as a non-treated group.

### 2. Preparation and Administration of Sample

100 mg and 200 mg of N-hydroxy-4-{5-[4-(5-isopropyl-2-methyl-1,3-thiazol-4-yl)phenoxy]pentoxy}-benzamidine were completely dissolved in 5 ml of sterilized distilled water. The benzamidine compound in the solutions was orally administered at doses of 100 mg and 200 mg per kg of body weight once a day from the day of the sensitization with ovalbumin. The control group was administered with equal volumes of sterilized distilled water in the same manner.

3. Asthma Induction by Immunization with and Exposure to Ovalbumin

A solution of 200 μg of ovalbumin (Grade V; Sigma, St. Louis, Mo., USA) and 180 mg of aluminum hydroxide (Al(OH)3, dried powder gel; Aldrich, Milwaukee, USA) in 4 ml of physiological saline was allowed to stand at 4° C. overnight and was administered to the experimental animals (200 μl, abdominal injection) for sensitization. As for the non-treated group, a solution of only aluminum hydroxide in saline was injected. 15 days after sensitization, a 1.5% ovalbumin solution was sprayed in air using a nebulizer, followed by exposing the experimental animals to the spray for 10 min to induce asthma therein. The non-treated group was exposed only to saline in the same manner. All the experimental animals were sacrificed 3 days after the exposure.

### 4. Change in Body Weight and Weight Gain

All the experimental animals were measured for body weight 1, 7, 14, 16 and 17 days after administration. In order to reduce difference among individuals due to feedstuff intake, all experimental animals were starved for 18 hours or more on the beginning day of the administration and on the sacrificing day before weight measurement. To minimize the difference of change in body weight of individual animals, body weight gains during time periods of the sensitization, the asthma induction after exposure and the whole experiment were calculated.

The results are given in Table 1, below.

TABLE 1
Change of body weight gain

| | | | Asthma induction | Over whole |
|---|---|---|---|---|
| | | Sensitization | period | experimental |
| Groups | | period | after exposure | period |
| Normal | | 3.08 ± 0.71 | 0.52 ± 0.33 | 2.34 ± 0.63 |
| Control | | 3.02 ± 0.84 | 0.84 ± 0.29 | 2.50 ± 0.73 |
| Cpd. of | 100 mg/kg | 3.50 ± 0.51 | 0.34 ± 0.34* | 2.68 ± 0.52 |
| Formula 1 | 200 mg/kg | 3.08 ± 0.71 | 0.36 ± 0.59 | 2.90 ± 0.40 |

*significance compared to control (p < 0.05)

As seen in Table 1, no significant changes in body weight gain were observed over all experimental periods except for the post-exposure asthma induction period, indicating that there are almost no errors attributable to the administration of experimental substances or the individual difference of experimental animals. Also in the post-exposure asthma induction period, the control group was observed to notably gain body weight whereas the benzamidine compound-administered group showed a remarkable decrease in weight gain.

### 5. Measurement of Lung Weight

On the final day of experiment, the lungs were separated from adjacent organs. The removed lungs were weighed in grams. To minimize errors due to the difference in body weight among individual animals, the relative weight of the lungs was calculated as a percentage of body weight using the following mathematic formula 1.

$$Relative Weight of Lung ( \% ) = \frac{Absolute Weight of Lung}{Body Weight} \times 100 \qquad Formula 1$$

The results are given in Table 2, below.

TABLE 2
Changes in Lung Weight

| Groups | | Absolute Wt.(g) | Relative Wt.(%) |
|---|---|---|---|
| Normal | | 0.112 ± 0.004 | 0.634 ± 0.021 |
| Control | | 0.138 ± 0.004* | 0.750 ± 0.015* |
| Cpd. Of | 100 mg/kg | 0.128 ± 0.007*, ## | 0.697 ± 0.030*, ## |
| Formula 1 | 200 mg/kg | 0.125 ± 0.008**, # | 0.682 ± 0.030**, ## |

Significance compared to normal (*p < 0.01, **p < 0.05), Significance compared to control(#: p < 0.01, ##: p < 0.05)

As is apparent from Table 2, the absolute and relative weights of the lungs according to asthma induction were significantly increased in the control group compared to the normal group (p<0.01) while they were significantly decreased in the benzamidine compound-administered group compared to the control group (p<0.01 or p<0.05) in a dose-dependent pattern.

Thus, the benzamidine compound of Chemical Formula 1 is found to prevent the weight of the lungs from increasing due to asthma.

### 6. Total Count of Leukocytes in Blood and Fractional Calculation of Leukocytes

On the final day of experiment, all the experimental animals were etherized and underwent laparotomy to expose the abdominal vena cava, from which 1 ml of blood was then taken. Using a hemocytometer, a blood sample was measured for total leukocyte counts in a ×10³/1 mm³ unit. Immediately after blood collection, the blood sample was smeared on slide glasses, fixed with methanol, and stained with Giemsa. And then, lymphocytes, eosinophils, neutrophils, monocytes, and basophils were calculated for their respective portions per 200 leukocytes and are represented as percentages in Table 3, below.

TABLE 3
Changes of Leukocyte Count in Blood

| Proportions | | | Benzamidine Cpd-administered | |
|---|---|---|---|---|
| Of Leukocytes | Normal | Control | 100 mg/kg | 200 mg/kg |
| Total | 8.24 ± 0.97 | 14.04 ± 0.91* | 10.68 ± 1.76**, ## | 10.44 ± 1.81**, ## |
| leukocytes | | | | |
| Lymphocytes(%) | 84.10 ± 1.98 | 40.00 ± 10.60* | 41.70 ± 12.90* | 53.80 ± 5.50*, # |
| Eosinophils(%) | 3.10 ± 1.39 | 54.20 ± 10.63* | 49.60 ± 15.79* | 36.90 ± 8.33*, # |
| Neutrophils(%) | 9.20 ± 2.00 | 3.80 ± 1.10* | 4.90 ± 2.90** | 5.20 ± 2.00** |
| Monocytes(%) | 3.20 ± 0.30 | 2.00 ± 1.20** | 3.70 ± 1.60 | 4.40 ± 1.70## |
| Basophils(%) | 0.30 ± 0.40 | 0.00 ± 0.00 | 0.10 ± 0.20 | 0.00 ± 0.00 |

significance compared to the normal (*p < 0.01, **p < 0.05), significance compared to the control (#: p < 0.01, ##p < 0.05)

According to asthma induction, the count of whole leukocytes in blood and its eosinophil proportion were, as can be understood from the data of Table 3, increased in the control group compared to the normal group, with significance (p<0.01), whereas they were decreased dose dependently in the benzamidine compound-administered group compared to the control group, with significance (p<0.01 or p<0.05).

Hence, this result indicates that the benzamidine compound of Chemical Formula 1 significantly suppresses the inflammatory response attributed to asthma.

### 7. Fractional Count of Cellular Components of Bronchoalveolar Lavage Fluid

On the final day of experiment, secretions present in bronchi and alveola were examined for cytological constitution. To this end, after being etherized, the experimental animals were operated to open the cervical region and the thorax. The jugular vein was allowed to bleed, followed by endotracheal intubation. 3 ml of phosphate buffered saline was injected twice through the tube and the thorax was massaged for 30 sec to obtain cell suspension from the lungs. The cell suspension was centrifuged at 3000 rpm for 30 min and resuspended in DPBS (Gibco BRL, NY, USA). After the cell suspension was smeared on a slide glass, the cells were stained with Giemsa. A total count of the whole cells present in the smear and fractional counts of neutrophils, eosinophils, basophils, macrophages, and epithelioid cells were measured and are given in Table 4, below.

TABLE 4
Changes of Leukocyte Count in bronchoalveolar lavage
fluid

| Proportions of | | | Benzamidine Cpd-administered | |
|---|---|---|---|---|
| Leukocytes | Normal | Control | 100 mg/kg | 200 mg/kg |
| Lymphocytes(%) | 59.90 ± 9.32 | 38.40 ± 12.30* | 56.90 ± 15.04 | 56.80 ± 22.52 |
| eosinophils(%) | 2.90 ± 0.74 | 14.60 ± 3.70* | 4.30 ± 1.44# | 3.60 ± 2.88# |
| Neutrophils(%) | 8.80 ± 0.91 | 16.20 ± 4.72* | 17.50 ± 8.06** | 13.90 ± 9.85 |
| Monocytes(%) | 3.80 ± 0.76 | 2.90 ± 2.82 | 3.10 ± 3.73 | 3.50 ± 2.57 |
| Basophils(%) | 0.70 ± 0.57 | 1.11 ± 0.42 | 0.50 ± 0.50 | 0.10 ± 0.22# |
| Epithelioids(%) | 22.40 ± 8.94 | 26.10 ± 16.91 | 21.00 ± 14.77 | 15.50 ± 9.74 |

significance compared to the normal group (*p < 0.01, **p < 0.05), significance compared to the control group (#p < 0.01, ##: p < 0.05)

According to Asthma induction, as seen in Table 4, the proportion of eosinophils in the bronchoalveolar lavage fluid was increased in the control group, compared to the normal group, with significance (p<0.01), but decreased in the benzamidine compound-administered group, compared to the control group, with significance (p<0.01) in a dose-dependent pattern.

Thus, it is found that the benzamidine compound of Chemical Formula 1 can remarkably restrain the inflammatory response induced by asthma.

### 8. Tissue Process and Histopathological Observation

The lungs removed after asthma induction were fixed in 10% neutral formalin and embedded in paraffin. The paraffin-embedded tissue was sliced at a thickness of 3 to 4 μm, stained with hematoxylin-eosin or Masson's trichrome and observed through an optical microscope.

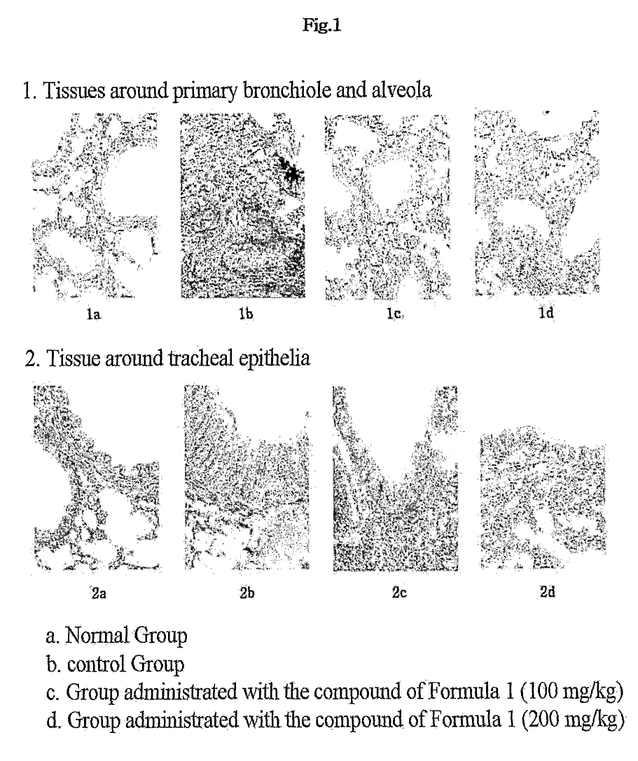

The results are given in FIG. 1.

According to asthma induction, the control group, as shown in FIG. 1, was found to have increased inflammatory cell populations in tissues around the primary bronchiole and alveola and around bronchial epithelia, as opposed to the normal group, but the benzamidine compound-administered group showed a reduction of the inflammatory cell population in the tissues in a dose-dependent pattern, compared to the control group.

From the lung tissue specimen prepared above, alveolar areas (proportion of alveolar lumen in lung tissue), populations of the goblet cells present in the bronchus and the bronchiole, and wall thicknesses of the bronchus and the bronchiole were examined using an analysis Image processing system (SIS Germany). The alveolar areas are represented as percentages, the populations of the goblets cells in the bronchus and bronchiole as counts per 1,000 cells, and the wall thickness of the bronchus and bronchiole in μm in Table 5.

TABLE 5
Change of histomorphology

| | | | | Benzamidine Cpd-administered | |
|---|---|---|---|---|---|
| | | Normal | Control | 100 mg/kg | 200 mg/kg |
| Alveolar Area | | 76.18 ± 4.68 | 26.80 ± 2.79* | 55.53 ± 4.26*, # | 61.62 ± 6.93**, # |
| Wall | Bronchus | 25.44 ± 3.79 | 124.34 ± 47.80* | 60.92 ± 8.53*, # | 40.69 ± 5.37*, # |
| Thick. | Brochiole | 16.91 ± 4.30 | 51.67 ± 11.11* | 27.73 ± 4.68*, ## | 24.79 ± 3.03*, # |
| Counts of | Bronchus | 139.00 ± 23.46 | 617.00 ± 87.01* | 317.60 ± 75.46*, # | 256.80 ± 71.58*, # |
| goblet | Brochiole | 17.60 ± 4.88 | 94.40 ± 26.89* | 53.80 ± 11.73*, ## | 42.80 ± 10.69*, # |
| Cells | | | | | |

significance compared to normal (*p < 0.01, **p < 0.05), significance compared to control (# p < 0.01, ## p < 0.05)

According to asthma induction, as seen in Table 5, the alveolar area of the lung tissue was decreased in the control group, compared to the normal group, with significance (p<0.01), but the benzamidine compound-administered group was found to have the alveolar area increased, compared to the control group, in a dose-dependent pattern, with significance (p<0.01).

As asthma was induced, the control group, compared to the normal group, was increased both in the wall thickness of the bronchus and bronchiole of the lungs and in the population of the goblet cells of the bronchus and bronchiole epithelium, with significance (p<0.01), but the benzamidine compound-administered group showed a significant decrease compared to the control group (p<0.01 or p<0.05), in a dose-dependent pattern.

As a result, the benzamidine compound of Chemical Formula 1 is identified to have a potent inhibitory effect on the inflammatory response attributed to asthma.

### 9. Statistics

All numerals are represented as mean±standard deviation, and statistical significance of the differences relative to the normal or the control was analyzed using Mann-Whitney U-Wilcoxon Rank Sum with the aid of SPSS (stastical package program for window) (Release 6.1.3., SPSS Inc., USA).

Likewise, methane sulfonate and hydrochloride of N-hydroxy-4-{5-[4-(5-isopropyl-2-methyl-1,3-thiazol-4-yl)phenoxy]pentoxy}benzamidine, and 4-{5-[4-(5-isopropyl-2-methyl-1,3-thiazol-4-yl)phenoxy]pentoxy}benzamidine and its methane sulfonate and hydrochloride were found to exhibit therapeutic effects similar to the above.

### INDUSTRIAL APPLICABILITY

The composition of the present invention can greatly reduce typical chronic inflammation symptoms, such as an increase of eosinophil level in bronchoalveolar lavage fluid, total leukocyte level and eosinophil level in blood, the hypertrophy or hyperplasia of bronchial epithelium due to an increase of mucus producing cells, a reduction in alveolar surface area resulting from the hypertrophy of alveolar walls, and the infiltration of inflammatory cells, thereby exhibiting excellent medicinal effects on allergic inflammatory diseases.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A pharmaceutical composition for the prevention and treatment of an allergic inflammatory disease, comprising a benzamidine compound, represented by the following chemical formula 1, or its pharmaceutically acceptable salt.

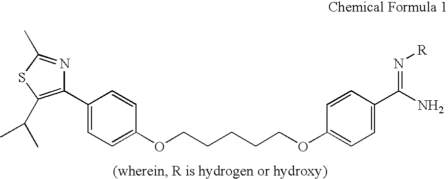

2. The pharmaceutical composition according to claim 1, wherein the salt is a methane sulfonate or hydrochloride.

3. The pharmaceutical composition according to claim 1, wherein the allergic inflammatory disease is selected from a group consisting of allergic rhinitis, asthma, allergic conjunctivitis, allergic dermatitis, atopic dermatitis, contact dermatitis and urticaria.

4. The pharmaceutical composition according to claim 3, wherein the inflammatory disease is asthma or allergic rhinitis.

5. A method of treating or preventing an allergic inflammatory disease, in which a benzamidine compound represented by the following chemical formula 1 or its pharmaceutically acceptable salt is administered to a patient with the allergic inflammatory disease: [chemical structure]
